(19) United States
(12) Patent Application Publication
(10) Pub. No.: US 2025/0169704 A1
(43) Pub. Date: May 29, 2025

(54) AUTOMATED CUFF INFLATION CONTROL FOR NON-INVASIVE BLOOD PRESSURE MONITORING

(71) Applicant: Covidien LP, Mansfield, MA (US)

(72) Inventors: Paul S. Addison, Edinburgh (GB); Dean Montgomery, Edinburgh (GB)

(21) Appl. No.: 18/928,611

(22) Filed: Oct. 28, 2024

Related U.S. Application Data
(60) Provisional application No. 63/603,008, filed on Nov. 27, 2023.

(51) Int. Cl. A61B 5/0225; A61B 5/00; A61B 5/01; A61B 5/0205; A61B 5/1455; A61B 5/291; G16H 10/60; G16H 40/63

(52) U.S. Cl. CPC A61B 5/0225; A61B 5/7267; A61B 5/7285; G16H 10/60; G16H 40/63; A61B 5/01; A61B 5/02055; A61B 5/14551; A61B 5/291; A61B 2560/0462; A61B 2562/0247; A61B 2562/0271

(57) ABSTRACT

A noninvasive blood pressure sensor may be activated based on data from one or more patient sensors, such as a pulse oximetry sensor. In an embodiment, when the sensor data is determined to be characteristic of or associated with a likely change in blood pressure, the noninvasive blood pressure sensor is activated to acquire a blood pressure measurement. A trained model may be used to determine a likelihood od the sensor dating being characteristic of or associated with the change in blood pressure.

### CROSS-REFERENCE TO RELATED APPLICATION

The present application claims the benefit of U.S. Provisional Patent Application Ser. No. 63/603,008, filed on Nov. 27, 2023, the entire content of which is incorporated herein by reference.

### TECHNICAL FIELD

The present disclosure relates generally to medical devices, and, more particularly, to systems and methods for monitoring blood pressure of a patient.

### BACKGROUND

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it may be understood that these statements are to be read in this light, and not as admissions of prior art.

In the field of medicine, doctors often desire to monitor certain physiological characteristics of their patients. In some cases, clinicians may wish to monitor a patient's blood pressure. Blood pressure may be assessed using a wide variety of monitoring devices. For example, blood pressure may be monitored non-invasively via a sphygmomanometer (e.g., a blood pressure cuff). A cuff, typically placed around the patient's arm, is inflated to a pressure that exceeds the systolic blood pressure, e.g., the pressure in the cuff is higher than the arterial blood pressure. Pressure can be reduced in the cuff until a pressure value at which blood begins to flow past the deflating cuff, indicative of the systolic pressure. The minimum pressure in the cuff that restricts blood flow is the diastolic pressure. Blood pressure measurements of a sphygmomanometer may be taken at scheduled intervals, because inflation of the cuff may be irritating for the patient.

### SUMMARY

Certain embodiments commensurate in scope with the originally claimed subject matter are summarized below. These embodiments are not intended to limit the scope of the disclosure. Indeed, the present disclosure may encompass a variety of forms that may be similar to or different from the embodiments set forth below.

In one embodiment, a noninvasive blood pressure monitoring system includes a noninvasive blood pressure sensor and one or more patient sensors that generate sensor data. The system also includes a monitoring device that receives the sensor data and that includes a memory storing a trained model and processing circuitry that provides the sensor data as input to the trained model and that generates a signal to activate the noninvasive blood pressure sensor based on an output of the trained model.

In another embodiment, a method of noninvasive blood pressure monitoring includes receiving sensor data of a patient from one or more patient sensors, the sensor data corresponding to a time window; determining, using a trained model, that the sensor data is associated with a predicted change in blood pressure for the patient; and triggering a noninvasive blood pressure sensor based on the predicted change in blood pressure.

Various refinements of the features noted above may exist in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended only to familiarize the reader with certain aspects and context of embodiments of the present disclosure without limitation to the claimed subject matter.

### BRIEF DESCRIPTION OF THE DRAWINGS

Advantages of the disclosed techniques may become apparent upon reading the following detailed description and upon reference to the drawings in which:

FIG. 1 is a perspective view of an embodiment of a medical monitoring system for noninvasive blood pressure monitoring with automatic cuff inflation, in accordance with an aspect of the present disclosure;

FIG. 2 is a block diagram of the medical monitoring system of FIG. 1, in accordance with an aspect of the present disclosure;

FIG. 3 is a flow diagram of a process of activating the cuff of the noninvasive blood pressure sensor of the medical monitoring system of FIG. 1, in accordance with an aspect of the present disclosure;

FIG. 4 is a flow diagram of a process of updated a model of the medical monitoring system of FIG. 1, in accordance with an aspect of the present disclosure;

FIG. 5 is a block diagram of an embodiment of the medical monitoring system having multiple patient sensors, in accordance with an aspect of the present disclosure;

FIG. 6 is a flow diagram of a process of activating the cuff of the noninvasive blood pressure sensor of the medical monitoring system of FIG. 5, in accordance with an aspect of the present disclosure;

FIG. 7 is a flow diagram of a process of selecting a model of the medical monitoring system of FIG. 5 based on patient sensor data availability, in accordance with an aspect of the present disclosure;

FIG. 8 is an example user interface of the medical monitoring system, in accordance with an aspect of the present disclosure;

FIG. 9 is an example user interface of the medical monitoring system, in accordance with an aspect of the present disclosure; and

FIG. 10 is an example user interface of the medical monitoring system, in accordance with an aspect of the present disclosure.

### DETAILED DESCRIPTION OF SPECIFIC EMBODIMENTS

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

Blood pressure monitoring may be used as part of vital signs monitoring and/or to identify hemodynamic instability of a patient. Blood pressure can either be obtained invasively, such as via an arterial catheter, or non-invasively. Invasive techniques may provide continuous real-time blood pressure measurements. However, arterial catheter insertion is complex, and clinicians may prefer using less invasive techniques. While continuous noninvasive blood pressure (NIBP) methods permit real-time blood pressure monitoring, continuous NIBP monitoring methods may be sensitive to patient movement and may be uncomfortable in extended use. Accordingly, clinicians may monitor blood pressure using intermittent NIBP techniques. In one technique, an NIBP sensor includes an occluding cuff that can be inflated using a manual trigger or at automatic intervals (e.g., every 15 minutes) to monitor patient blood pressure. However, changes in blood pressure that occur between monitoring points may be missed. Further, if the patient's blood pressure is relatively stable, such regular monitoring may not be necessary. Because the cuff of the NIBP sensor is inflated to pressures that occlude blood flow and then deflated over time, the inflation and subsequent deflation process may be distracting for the patient. Thus, it may be beneficial to avoid taking blood pressure measurements while blood pressure is unchanging.

The present disclosure is directed to systems and methods for activating or triggering a sensor of an NIBP monitoring system such that the sensor is likely to be triggered concurrently with changes in blood pressure. The disclosed techniques acquire data from another patient monitoring sensor (or sensors) applied to the patient to determine if the sensor data is associated with a likely change in blood pressure. This is achieved using a trained artificial intelligence (AI) model to identify characteristic patient sensor data and/or metrics derived from such data that are associated with concurrent changes in blood pressure. Sensor data (e.g., real-time sensor data, sensor data over a particular time window) from ongoing monitoring of a patient is used as input to the trained model. The trained model in turn generates an output of a likelihood of a change in blood pressure that is associated with the input sensor data. If the likelihood is high, based on thresholds set by the system and/or operator, a trigger signal is generated. The trigger signal causes a cuff of the NIBP sensor to inflate to initiate acquiring blood pressure for the patient using the NIBP sensor. The NIBP sensor is not activated when the likelihood is below the threshold. Thus, rather than having the NIBP sensor activated at regular timed intervals, the NIBP sensor activation is variable and responsive to updating sensor data from one or more other patient sensors.

In one embodiment, the acquired blood pressure can be fed back into the trained model to assess if the model-determined likelihood of a change in blood pressure was accurate for a particular cuff trigger event. The measured blood pressure from the cuff trigger can be provided as labelled truth to the trained model. If the measured blood pressure changed relative to a baseline (e.g., the most-recent previously acquired blood pressure or a window of recent measured blood pressures) by at least a threshold amount, the trigger event can be marked as accurate, which can increase a confidence for the particular modeled likelihood. If the measured blood pressure did not change relative to the baseline by the threshold amount, the trigger event can be marked as an error, which in turn can decrease the confidence for that particular modeled likelihood. In this manner, the trained model can be dynamically updated to be retrained on particular patient. Over time, as incoming patient data and associated blood pressure triggers are fed into the trained model, the trained model becomes more patient-tailored.

The disclosed techniques provide improved detection of rapid and/or variable changes in blood pressure and detection of otherwise missed blood pressure events while avoiding triggering inflation of the cuff of the NIBP sensor while patient blood pressure is relatively stable. For patients with relatively stable blood pressure, this may result in less frequent activation of the NIBP, which may be more comfortable. For patients with changing blood pressure, this may result in earlier identification of blood pressure changes relative to arrangements in which the blood pressure is measured at preset intervals.

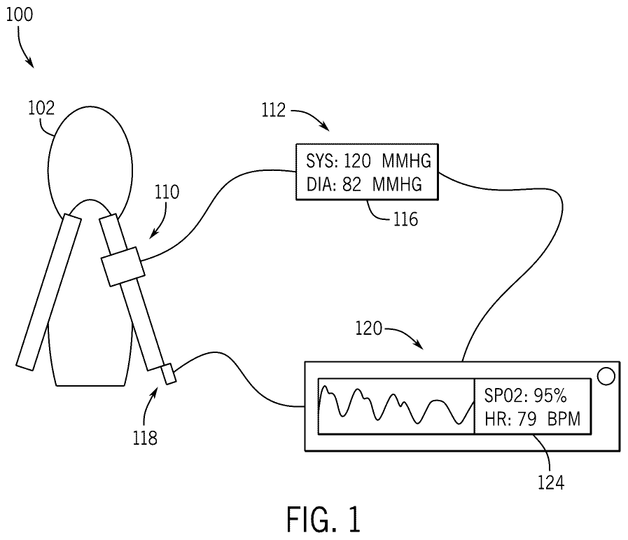

FIG. 1 illustrates a schematic view of an embodiment of a noninvasive blood pressure (NIBP) monitoring system 100 as provided herein for monitoring physiological parameters of a patient 102. The system 100 includes a NIBP sensor 110 that is coupled to a NIBP monitor 112. The NIBP monitor 112 displays blood pressure measurements 116 based on data from the NIBP sensor 110. Blood pressure measurements 116 may computed from one or more received signals (inflatable cuff pressure sensors) and may include, for example, systolic blood pressure, diastolic blood pressure, or both, or any suitable manipulation of any suitable blood pressure value. In some embodiments, blood pressure measurements 116 may be the output of one or more mathematical operations to generate blood pressure parameters or one or more parameters derived therefrom (e.g., blood pressure variability, range, maximum, minimum, etc.).

In addition, the system 100 includes one or more additional patient sensors, illustrated here as a pulse oximetry sensor 118. In the illustrated example, the patient 102 is undergoing NIBP monitoring and additional physiological parameter monitoring concurrently. The pulse oximetry sensor 118 is coupled to a pulse oximetry monitor 120 that determines and/or displays physiological parameter measurements 124, such as a photoplethysmography (PPG) signal, an oxygen saturation (SPO₂) value, and/or a heart rate value as in the illustrated example. In addition, the system may also determine derived values such as pulse rate, pulse wave area, dichrotic notch position, skew of a portion of a PPG signal, skew of the derivative of a portion of a PPG signal, upstroke length of a pulse wave, pulse wave height, pulse wave full width at half maximum (FWHM), any other suitable metric, or any suitable combinations thereof.

The physiological parameter measurements 124 may include raw data from the pulse oximetry sensor 118 as well as the calculated parameters as discussed. The physiological parameter measurements 124 may be used to generate a trigger signal for the NIBP sensor 110. Thus, the NIBP monitor 112 and the pulse oximetry monitor 120 may be communicatively coupled such that the physiological parameter measurements 124 and/or a generated trigger signal can be communicated to the NIBP monitor 112. The communication may be wired or wireless in embodiments. In certain embodiments, the trigger signal may be determined on the pulse oximetry monitor 120 using a trained model, as generally discussed herein, that is stored on and executed on the pulse oximetry monitor 120. Thus, the model output may be a trigger signal that is communicated from the pulse oximetry monitor 120 to the NIBP monitor 112. In an embodiment, physiological parameter measurements 124 are communicated to the NIBP monitor 112, which stores and executes the trained model using the incoming physiological parameter measurements 124.

Automatic cuff control (e.g., NIBP sensor activation) using data from a separate patient sensor may be an add-on function of the system 100 that is active when sensor data from a compatible sensor is present. In an embodiment, the function may be activated based on a coupling of a cable between the pulse oximetry monitor 120, or other monitor, to the NIBP monitor 112, at an appropriate input port, based on incoming compatible sensor data at the NIBP monitor 112, and/or based on an incoming trigger signal at the NIBP monitor 112. The pulse oximetry sensor 118 and the NIBP sensor 110 may communicate in the system 100 using wired or wireless connections.

It should be understood that the illustrated blood pressure measurements 116 and/or physiological parameter measurements 124 are by way of example, and additional or other information may be determined and/or displayed by the system 100. Further, while the NIBP monitor 112 and the pulse oximetry monitor 120 are illustrated as separate, standalone devices, it should be understood that the NIBP monitor 112 and the pulse oximetry monitor 120 may be integrated or combined as a multiparameter monitor in certain embodiments. Thus, in such an embodiment, communication between these devices may be achieved using internal device communication. In certain embodiments, the pulse oximetry sensor 118 and the NIBP sensor 110 are separate devices that can be independently applied to separate locations on the patient 102. For example, the NIBP sensor 110 can be applied to a patient arm while the pulse oximetry sensor 118 can be applied to the patient finger. However, in certain cases the patient sensor, e.g., the pulse oximetry sensor 118, and the NIBP sensor 110 may be integrated into a unitary device.

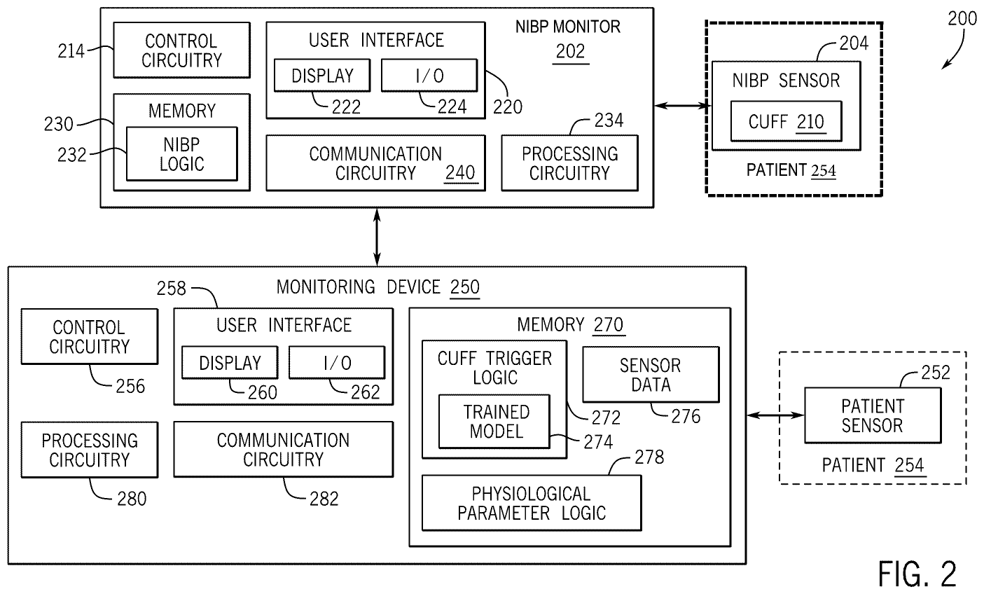

Turning to FIG. 2, a simplified block diagram of a medical monitoring system 200 (e.g., the medical monitoring system 100 of FIG. 1) is illustrated in accordance with an embodiment. The system 200 includes an NIBP monitor 202 that is coupled to and that controls an NIBP sensor 204. The NIBP sensor 204 includes an inflatable cuff 210. Inflation and deflation of the cuff 210 is controlled via control circuitry of the NIBP monitor 202. The NIBP sensor 204 may include a pressure transducer in communication with the cuff 210. A signal from the pressure transducer may be passed to processing circuitry 234 to determine blood pressure measurements using NIBP logic 232 stored in memory 230. The NIBP logic 232 may receive sensor data from the pressure transducer indicative of pressure oscillations in the cuff 210. The NIBP monitor 202 may control inflation of the cuff 210 with a pump (not shown) to execute an inflation and subsequent deflation, e.g., an inflation pattern, of the cuff 210 as part of blood pressure monitoring. For example, the NIBP monitor 202 may activate a motor of the pump to transfer fluid (e.g., ambient air) into the cuff 210 to increase a cuff internal pressure. The NIBP monitor 202 may vent air from the cuff 210 using a valve to decrease a cuff internal pressure.

The NIBP monitor 202 may include user interface 220 having a display 222 and one or more input/output devices 224 (e.g., keyboards, input keys, touchscreen, speakers). The user interface 220 and/or other user interfaces as provided herein present information to a user (e.g., a clinician). In some examples, user interface 220 may be part of a multiparameter monitor (MPM) or other physiological signal monitor used in a clinical or other setting, a personal digital assistant, mobile phone, tablet computer, laptop computer, any other suitable computing device, or any combination thereof, with a built-in display or a separate display.

In some examples, the NIBP monitor 202, via communication circuitry 240, may receive input signals from a coupled monitoring device 250. The monitoring device may in turn be coupled to or control the operations of one or more patient sensors 252 monitoring physiological parameter/s of a patient 254 who is also undergoing monitoring with the NIBP sensor 204. The sensor 252 may be a blood pressure sensor, pulse oximetry sensor, regional oxygen saturation sensor, regional cerebral oxygen sensor, blood volume sensor, heart rate sensor, temperature sensor, electrocardiogramansor, electroencephalogram (EEG) sensor, a capnography sensor, or any combination thereof.

The monitoring device 250 may include control circuitry (e.g., processing circuitry 256), a user interface (e.g., user interface 258) have a display (e.g., display 260) and one or more input/output devices (e.g., input/output 262), memory (e.g., memory 270), processing circuitry (e.g., processing circuitry 280), and communication circuitry (e.g., communication circuitry 282). The memory 270 stores sensor data 276 from the patient sensor 252 and uses the sensor data to calculate physiological parameters via physiological parameter logic 278. In an embodiment, the memory 270 also stores cuff trigger logic 272 that includes a trained model 274. The trained model 274 operates on the sensor data 276 to generate a trigger signal that is output to the NIBP monitor 202 to activate inflation of the cuff 210. While the cuff trigger logic 272 is illustrated as being part of the monitoring device 250, it should be understood that the cuff trigger logic 272 may be included in the NIBP monitor 202. In such an embodiment, the sensor data 276 is communicated from the monitoring device 250 to the cuff trigger logic 272 of the NIBP monitor 202. As disclosed herein, the sensor data 276 may be provided to the cuff trigger logic 272 of the system 200 as one or both of raw data (e.g. the detected PPG signal) or calculated parameters or features based on the raw signal. Further, the NIBP monitor 202 and the monitoring device 250 may be integrated into a single device with certain overlapping elements eliminated.

The processing circuitry 234, 280, as well as other processors, processing circuitry, controllers, control circuitry, and the like, described herein, may include one or more processors. The processing circuitry 234, 280 may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, processing circuitry 234, 280 may include multiple components, such as any combination of one or more microprocessors, one or more DSPs, one or more ASICs, or one or more FPGAs, as well as other discrete or integrated logic circuitry, and/or analog circuitry. Certain function of the processing circuitry 234, 280 may be performed using a distributed or cloud computing environment.

The memory 230, 270, or other memory disclosed herein, may store program instructions, such as algorithms to execute the trained model 274 to generate an output, algorithms to calculate physiological parameter measurements, algorithms to update user interfaces 220, 258, algorithms to trigger notifications or alarms. The program instructions may include one or more program modules that are executable by the processing circuitry 234, 280. When executed by processing circuitry 234, 280, such program instructions may cause the processing circuitry 234, 280 to provide the functionality ascribed to it herein. The program instructions may be embodied in software, firmware, and/or RAMware.

The communication circuitry 240, 282 may permit communication between the NIBP monitor 202 and the monitoring device 250 as well as communication with other devices coupled to the system 200. Communication may be direct communication or may be passed through one or more computing devices, such as one or more non-edge switches, routers, hubs, dongles, adapters, gateways, security devices such as firewalls, intrusion detection, and/or intrusion prevention devices, servers, computer terminals, laptops, printers, databases, wireless mobile devices such as cellular phones or personal digital assistants, wireless access points, bridges, cable modems, application accelerators, or other network devices. Communication techniques may include, for example, communication according to the Bluetooth® or BLE protocols. The communication circuitry 240, 282, may include input circuitry to receive a signal from another device of the system 200. In some examples, the communication circuitry 240, 282 may include output circuitry to transmit information to another device of the system 200.

The communication circuitry 240, 282 may send information to another device on a continuous basis, at periodic intervals, upon request from another device, or based on processor-based instructions. The communication circuitry 240, 282 may also send a command to another device. The communication circuitry 240, 282 may send a trigger signal to cause activation (e.g., cuff inflation) of the NIBP sensor 204. The communication circuitry 240, 282 may send patient sensor data 276 or a request for patient sensor data 276.

The processing circuitry 234, 280 may operate to modify a raw signal from a sensor (e.g., the NIBP sensor 204 or the patient sensor 252) by, for example, filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof. In some examples, the processing circuitry 234, 280 may perform any suitable digital conditioning of the converted digital signals, such as low pass, high pass, band pass, notch, averaging, or any other suitable filtering, amplifying, performing an operation on the signal, performing any other suitable digital conditioning, or any combination thereof. The processing circuitry 234, 280 may operate to calculate physiological parameters from sensor data that may include derivatives of any suitable order, integrals, statistics (e.g., mean, standard deviation, skew, kurtosis), regressions of any form and order, ordinate values, abscissa values, any other suitable metrics, evaluated at any suitable point or combination of points, which may be computed from a suitable signal, or any suitable combinations thereof.

For example, in cases in which the patient sensor 252 is a pulse oximetry sensor, the sensor data 276 includes a PPG signal that may be used to determine oxygen saturation values and heart rate values as well as features such as an amplitude or frequency of an AC or DC component of the PPG signal, a percent modulation (indicative of changes in vasotone), a ratio of a DC IR component over a DC red component (indicative of changes in venous volume), and/or a skew of the derivative of the PPG signal (indicative of vascular resistance; related to left ventricular ejection time). Other PPG features may include metrics of the AC components of the red and IR signals, amplitudes, frequencies, and/or areas under the curve for pulses of the pulse waveform, times between fiducial points on the pulse waveform, the curvature of the pulse waveform including around the dichrotic notch, etc. These metrics may be calculated using the original PPG signal and/or its derivative, second derivative, etc. Further, ratios or derived combined indexes of these features are also contemplated. It should be understood that these features are by way of example. The sensor data 276 and/or features determined from the sensor data may be provided as inputs to the trained model 274.

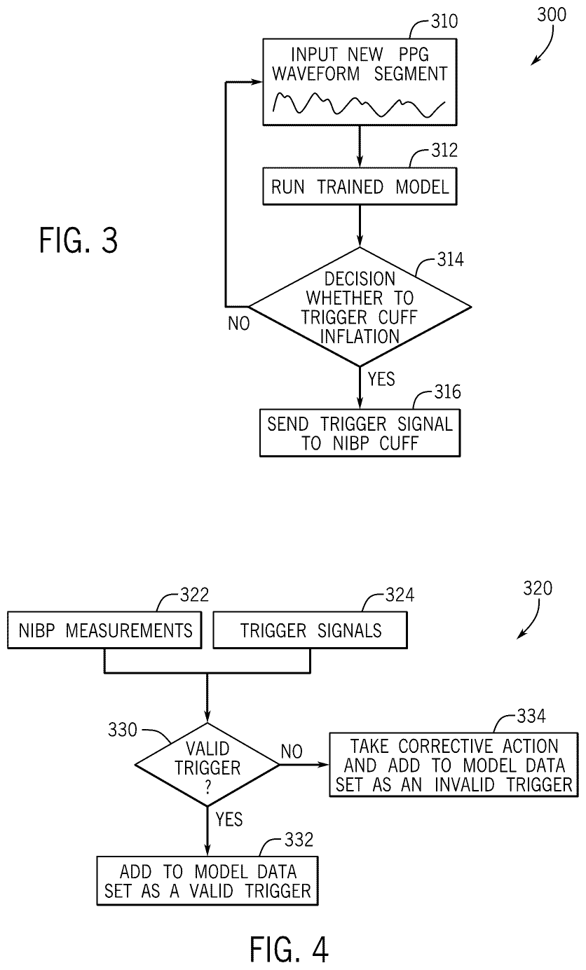

FIG. 3 is a flow diagram of an example method 300 of NIBP monitoring using a trained model that operates on sensor data and/or related sensor data features. Although FIG. 3 is discussed with respect to certain features of FIGS. 1-2, in other examples, different devices or device components, may perform any part of the method 300. For example, the method 300 may use any type of sensor and sensor data as discussed herein.

In the illustrated embodiment of the method 300, sensor data (e.g., sensor data 276) is acquired from a patient sensor (e.g., patient sensor 252) coupled to a patient (e.g., patient 254) that is also undergoing NIBP monitoring. The sensor data may be from a pulse oximetry sensor (e.g., pulse oximetry sensor 118) and may include a PPG signal. For each iteration of the method 300, a new PPG waveform segment, representative of a time window, can be provided as input to a trained model (e.g., trained model 274) in block 310. The PPG waveform segment may be a raw PPG signal(s) provided as the input in on embodiment. The trained model is run on the input data at block 312. At decision block 314, the model provides an output as to whether to trigger cuff inflation of an NIBP sensor (e.g., inflation of the cuff 210 of NIBP sensor 204). If the decision is to trigger the cuff inflation, a trigger signal is communicated to cause inflation of the cuff. As discussed herein, the trigger signal may be generated from a coupled monitor, such as the monitoring device 250, and communicated to an intervening device, such as the NIBP monitor 202, which in turn passes the trigger signal to the cuff 210. In other embodiments, the trigger signal may be generated on the device that controls the NIBP sensor 204, such as the NIBP monitor 202.

The output of the model may be, in an embodiment, 1) generate a trigger signal or 2) no trigger signal. The classification may be based on a likelihood of the sensor data being associated with a change in blood pressure. The model may be trained on data sets including labeled truth blood pressure changes and associated sensor data conditions. The trained model 274 may output values indicative of the likelihood that input data is associated with a change in blood pressure. For example, an output value of the model may be “80% likelihood of a change in blood pressure.” The monitoring system (e.g., monitoring system 100, 200) may apply configurable thresholds (e.g., above 50%, 75%, 90%, 95%, or 99%) to the likelihood values to classify the data and, in turn to determine with the output is associated with a “YES” to trigger cuff inflation or a “NO” to no trigger cuff inflation. For example, all likelihood values for predicted changes above a certain likelihood percentage may cause a trigger signal to be generated, while likelihood values below the certain percentage may cause no trigger signal.

Further, the likelihood of the change may be a likelihood of a change relative to an absolute threshold for one or both of systolic or diastolic blood pressure measurements. For example, if a previous measurement was within a normal range relative to a blood-pressure related alarm trigger, a likely change may be defined as a predicted movement of a most-recent measured systolic blood pressure and/or a diastolic blood pressure to an alarm-triggering value range from a normal range. Thus, any highly likely predicted movement from a normal range to an alarm-triggering range may cause the model to output a high likelihood of change and, therefore, generate a trigger signal. In an embodiment, a normal blood pressure range may be considered to be systolic values of less than 120 mm Hg and/or diastolic values of less than 80 mm Hg, and an alarm-triggering range may be outside of that. In an embodiment, a normal blood pressure range may be considered to be systolic values between 60 mm-120 mm Hg and/or diastolic values between 40-80 mm Hg, and an alarm-triggering range may be outside of that. However, it should be understood that these ranges may be set by a caregiver according to patient age and/or clinical condition.

In an embodiment, predicted changes that are below a certain threshold percentage threshold are not considered likely changes to avoid generating trigger signals for small variabilities. For example, if a measured systolic pressure is 100 mm Hg, a predicted change to 102 mm Hg (a 2% change) may not be considered a sufficient change to generate a trigger signal by the model having a 5% change threshold, while a predicted change from 100 mm Hg to 106 mm Hg (a 6% change) will generate a trigger signal in an embodiment. If no change is predicted, the predicted blood pressure may be classified as likely to be stable.

It should be understood that the method 300 and the PPG segment is by way of example, and other or additional data may be provided as input to the trained model as discussed herein. For example, the patient sensor may generate a characteristic signal of another type (e.g., EEG, capnography, respiration) that can be provided as a signal segment to the trained model. As discussed, the model may additionally or alternatively use calculated metrics or parameters derived from the sensor data in the classification.

In some examples, the trained model is trained with historical or previously acquired training data from plurality of patients that is truth-labeled with descriptive flags. For example, a training data set may include truth-labeled blood pressure data (e.g., from arterial or continuous blood pressure monitoring) and associated patient sensor data. For complex training data with multiple features and/or sensor data types, the training data may be processed into vectors and multi-dimensional arrays upon which monitoring system may apply mathematical operations, such as linear algebraic, nonlinear, or alternative computation operations. The monitoring system uses the training data to train machine learning models to weigh different features and/or to apply different coefficients when evaluating data to identify sensor data that is associated with a concurrent change in blood pressure or that is a leading indicator of a subsequent change in blood pressure. While certain techniques may use data metrics or thresholds applied to sensor data, the present techniques use machine learning to permit more variable data inputs and change in blood pressure identification.

The trained model may, for example use suitable machine learning method. For a feature based approach, a classifier such as a k-NN, SVM, Decision Tree, Random Forest, Neural Network etc., may be used. For a deep learning approach, a CNN based approach, a Transformer, or an LSTM model to perform the classification may be used. The model architecture may include a deep learning architecture such as a recurrent neural network, convolutional neural network, and the like that includes multiple layers to progressively extract higher level features from inputs to the trained model.

After training, the monitoring system may receive data for a particular patient. The monitoring system applies the model to the sensor data to determine whether to trigger a cuff of the NIBP sensor to initiate blood pressure monitoring. In an embodiment, the model may compare an input PPG signal segment from a time window immediately subsequent to a last iteration of the method 300. That is, the segment can represent a time window extending from a last run of the model to the current time point. While the disclosed methods permit cuff control at variable intervals based on the model output, the model itself may be run to a classification prediction at a regular interval. For example, the method 300 can be iterated every 1, 2, 5, 10, etc. seconds to generate a decision if the cuff should be inflated to take a blood pressure measurement. Thus, in an embodiment, the method 300 may use a fixed segment size as model input. Further, in embodiments in which additional or other signal-derived parameters or features are provided to the model, the signal-derived parameters or features may be based on a signal segment corresponding to a particular time window.

As provided herein, a trigger signal that activates cuff inflation may refer to activation of the cuff inflation pattern, which may encompass all or part of an inflation pattern including initial inflation of the cuff past systolic pressure (e.g., 150 mmHg-200 mmHg) and subsequent controlled deflation to determine systolic and diastolic pressures. Thus, a cuff that is activated via a trigger signal may be inflated from a resting inflation pressure or uninflated state and may be activated to inflate/deflate at various pressure of an inflation pattern. After the inflation pattern is executed, the cuff 210 may return to the baseline resting state or uninflated state after the activation by the trigger signal. The system may include cool-down periods to ensure that the cuff does not activate too frequently-for example-no more than once every 1, 3, 5 or 10 minutes.

FIG. 4 is a flow diagram of a method 320 to monitor the accuracy of model predictions. The method 320 may operate concurrently with application of the model as discussed herein. Thus, the present techniques may include real-time and/or background quality checks for the model. Turning to the method 320, the system (e.g., the system 100, 200) has access to the blood pressure measurements 322 after a measurement has been triggered based on a trigger signal 324. Each blood pressure measurement 322 that is based on a trigger signal 324 is evaluated to identify if the trigger signal is valid at block 330. If the trigger signal is valid, the associated sensor data that generated the model output of the trigger signal is added to the model dataset as a valid trigger as part of a training set at block 332. If the trigger is invalid, the associated sensor data that generated the model output of the trigger signal is added to the model dataset as an invalid trigger as part of a training set at block 334.

Assessment of a validity of the trigger signal may be based on whether a change in blood pressure relative to a previous measurement or relative to an absolute threshold is present in the NIBP measurement acquired as a result of the trigger signal. If a true change is present, the trigger signal is marked as valid, and if a change is not present, the trigger signal is marked as invalid. The assessment of the true change may be as discussed herein (e.g., blood pressure changes above a threshold, blood pressure measurements that move into an alarm-triggering range). In addition, the identification of one or more invalid triggers during monitoring may result in corrective action at block 334. For example, if the system repeatedly activates the cuff but the measurement of blood pressure indicates no change, the model may be poorly performing model for a particular subject. In an example, the system may take some corrective actions, such as increasing the confidence or likelihood threshold required to trigger the measurement (e.g. from a 50-70% threshold to an 80-90% threshold). The system may additionally or alternatively disable the model entirely and activate a regular timed interval schedule for measurements in cases where the model is flagged as poorly performing. In an embodiment, a threshold number of identified invalid triggers may cause the corrective actions. Further, the model could continue to be run in the background to check performance and allow re-enablement at a subsequent point.

The model may permit real-time retraining to change or update the model on data from an actively monitored patient. As inputs to the model are already collected and the labelled truth is the result of the cuff measurement, the model can be retrained over time to produce a patient-tailored model. For example, in certain cases, variable sensor placement on a patient may lead to input sensor data that is not aligned with the training set for the model. For example, sensor data from a well-placed sensor may yield different signal features than sensor data from a poorly adhered or shifting sensor. However, over time, even data from a poorly placed sensor can be evaluated for likely blood pressure changes using the model. Adjustments to the machine learning algorithm of the trained model may include adjusting weights and/or connections between nodes, as examples.

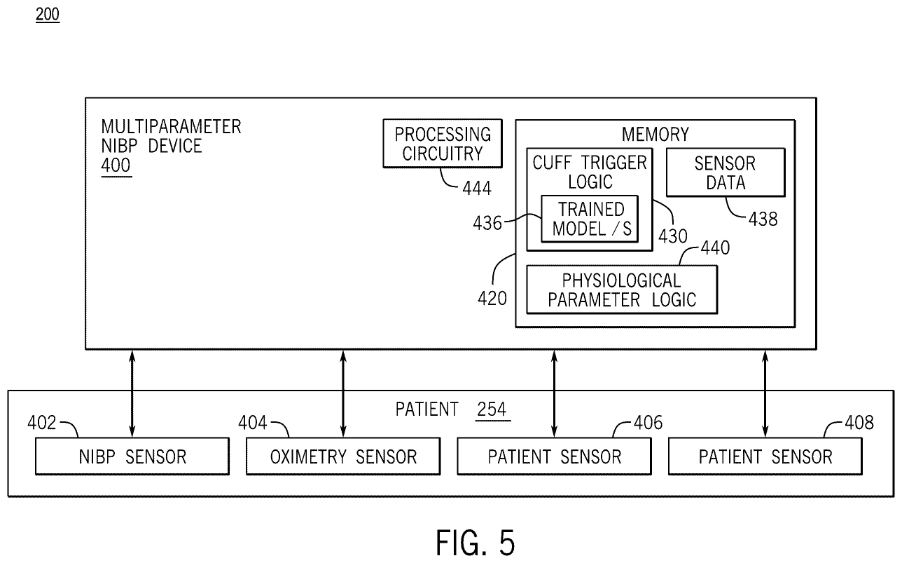

FIG. 5 is an example simplified block diagram of the monitoring system 200 including a multiparameter NIBP monitoring device 400 that is an integrated device instead of the separate devices illustrated in FIGS. 1-2. Certain features of the system 200 discussed with respect to FIGS. 1-2, although not shown, may be included in the illustrated NIBP monitoring device 400. Further, the arrangement and implementation of the features NIBP monitoring device 400 may be as discussed with respect to FIGS. 1-2.

The NIBP monitoring device 400 may couple to one or more patient sensors monitoring the patient 254, including the NIBP sensor 402 and one or more additional patient sensors, such as an oximetry sensor 404 and/or additional sensors 406, 408 by way of example. The sensors 406, 408 may be one or more of any medical monitoring sensor, e.g., as discussed herein. It should be understood that the NIBP monitoring device 400 may be capable of coupling to more or fewer sensors. In an embodiment, the NIBP monitoring device 400 may be a RespArray™ device (Medtronic), which may have sensor data from one or more of pulse oximetry, ECG, CO₂, temperature, etc.

The memory 420 of the multiparameter NIBP monitoring device 400 stores cuff trigger logic 430 to control activation of the coupled NIBP sensor 402 as discussed herein using the sensor data 438 from coupled or available sensors. Thus, the sensor data 438 may be provided to one or more trained models 436 as well as to physiological parameter logic 440 to permit the NIBP monitoring device 400 to perform normal monitoring functions. The NIBP monitoring device 400 may operate under control of processing circuitry 444.

The NIBP monitoring device 400 may have available input circuitry to permit the caregiver to select the desired patient sensors and to omit any sensors that are not of interest. Thus, the configuration of sensors coupled to the NIBP monitoring device 400 may be variable. Accordingly, the cuff trigger logic 430 may identify incoming sensor data 438 and apply model/s 436 to the sensor data 438 as appropriate. In an embodiment, the identified sensor data 438 may be identified based on an encoder or other identifying information provided from the coupled patient sensor/s that provides information about a type of sensor to the NIBP monitoring device 400.

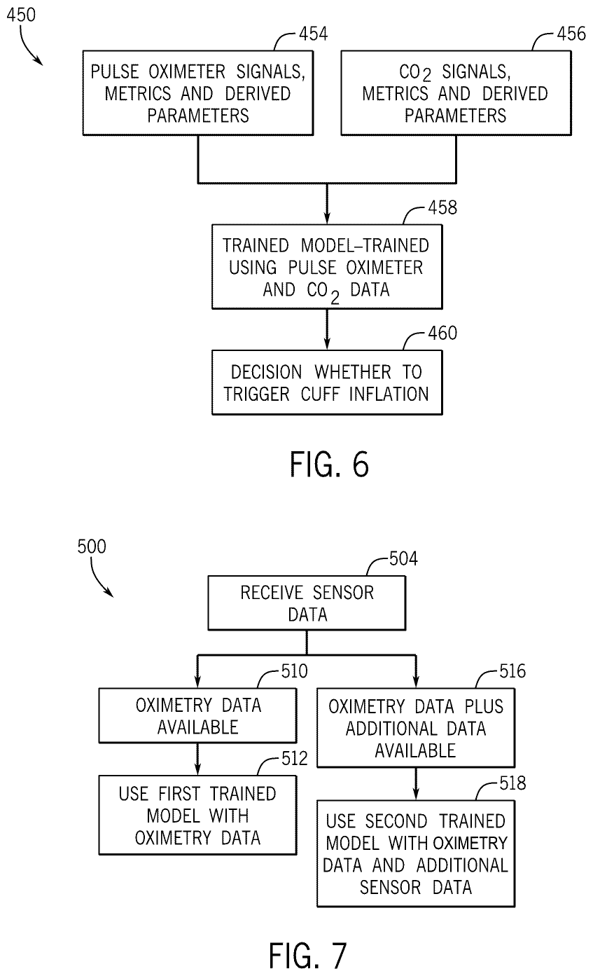

FIG. 6 is a flow diagram of a method 450 in which the monitoring system (e.g., monitoring system 100, 200) receives pulse oximetry sensor data 454 and other sensor data, such as capnography data (CO₂) 456. In some embodiments, only capnography data (CO₂) from a capnography sensor may be available, and not pulse oximetry sensor data 454. In other embodiments, only pulse oximetry data is available. The data 454, 456 is provided to the trained model at block 458 to generate a decision whether to trigger cuff inflation at block 460.

In certain cases, the system may consider certain sensor data to be unavailable if the sensor data falls below a quality threshold. Further, the system may arbitrate between different parameters that may be generated from different sensors. For example, if an EEG sensor has a determined heart rate with a higher confidence than a heart rate determined from a pulse oximetry sensor, the system may select the higher-confidence parameter as input to the model.

FIG. 7 is a flow diagram of a method 500 in which the monitoring system (e.g., monitoring system 100, 200) is capable of selecting between available trained models based on the incoming sensor data. At block 504, a monitoring device of the system receives sensor data and determines which data is available to provide to the trained model. In the illustrated example, if oximetry data, and not additional compatible data is available at block 510, the method 500 uses a first trained model with the incoming sensor data at block 512. If oximetry data plus additional compatible sensor data is available at block 516, the method uses a second trained model with the oximetry data and additional data at block 518.

For example, the first model may be a base or default model that uses only oximeter information as the input, and the second model may incorporate one or more of the ECG, CO₂, temperature, etc. These models may provide increased performance and be activated when these other parameters are available to the user. It should be understood that other model arrangements may be available that use the availability of other sensor data (e.g., CO₂) or any combination of sensor data as a base model and additional or other sensor data to operate enhanced models.

In this manner, the disclosed monitoring systems can provide automatic cuff triggering for a variety of different healthcare arrangements. For example, patient being monitored in a home environment may have a more limited set of applied sensors while patients being monitored in a hospital setting may be monitored by multiple sensor types.

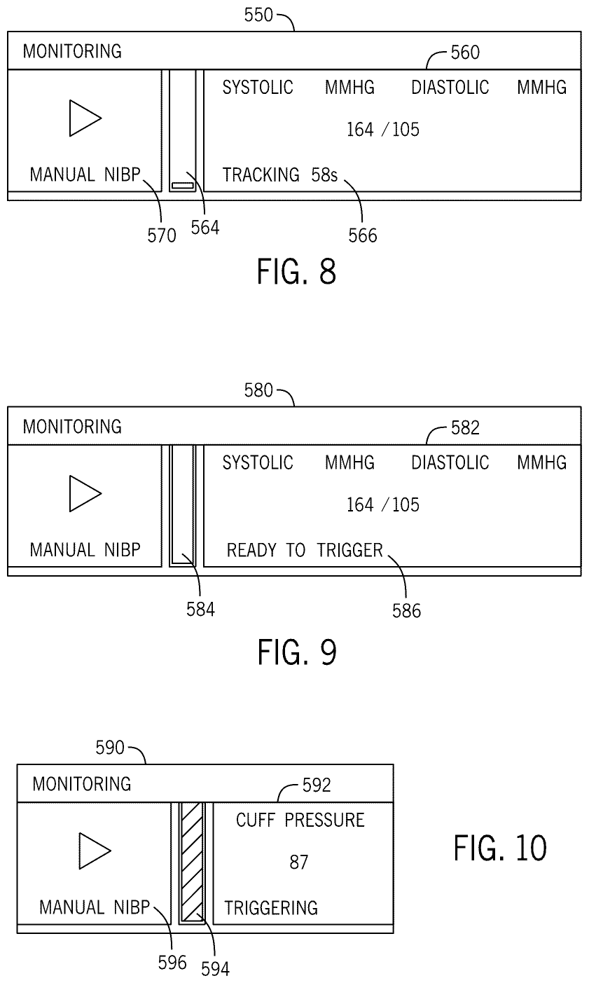

FIGS. 8-10 show different example graphical user interfaces or portions of graphical user interfaces of the monitoring system that maybe used in conjunction with the disclosed embodiments. In FIG. 8, an example user interface 550 shows monitored blood pressure values 560. These values 560 may represent a last-measured or most-recently measured value. In addition, the interface 550 may include an updating graphical icon 564 that fills during a cuff cool-down period. The cool-down period may be a feature to prevent the cuff from being activated within this period to avoid too-frequent activation from disrupting patient rest. The cool-down period within a preset time interval (e.g., 1, 3, 5 or 10 minutes). The preset cool down interval can be adjusted by an operator based on patient characteristics. In embodiments, the cool-down period can be automatically set based on model outputs. For example, if the incoming and/or recent sensor data is indicative of a relatively stable patient status, the cool-down period can be set to a longer interval (e.g., 10 minutes). If the updated sensor data shows a change in stability, the cool-down period can be automatically shortened (e.g., changed from 10 minutes to 5 minutes or 1 minute). That is, in the illustrated example, the icon 564 may be in the form of a bar that is empty at the beginning of the cool-down period and that fills as the cool-down period expires. The illustrated icon graphical 564 is by way of example, and other configurations may include different shapes (e.g., circle, triangle) or different changing graphical elements. For example, the graphical icon 564 may empty instead of fill over time. In an embodiment, the user interface may include an updating text icon 566 showing a time remaining until the cool-down period is expired blood pressure value can be measured again by automatic triggering of the cuff. The user interface 550 may include an override manual NIBP activator 570 to permit the operator to manually acquire blood pressure outside of the automatic system settings.

FIG. 9 is an example of an updated user interface 580 after expiration of the cool-down period. The tracking icons have updated configurations 586, 584 indicating graphically and/or via text that the cuff can be automatically triggered or is in a state of readiness. Whether or not the cuff is triggered is based on model output, as disclosed herein. That is, the expiration of the cool-down period itself does not trigger the cuff inflation. Rather, patient sensor data that is being concurrently acquired is provided to the trained model, which will in turn generate a model output. For model outputs that are associated with a low or below threshold likelihood of a change in blood pressure, no trigger signal is generated. Thus, as shown in the user interface, the blood pressure measurements 582 that are displayed may represent the last-measured values, as shown in FIG. 8. When the model output is indicative of a likelihood of a change in blood pressure, cuff inflation is triggered. FIG. 10 shows an example user interface 590 during a cuff triggering event. The blood pressure values are replaced with a cuff pressure indicator 592, which may display an internal cuff pressure indicative of a time point in the cuff inflation pattern. The displayed configuration of the tracking icon/s 594 updates to reflect active cuff trigging. In the illustrated embodiment, the fill pattern of a graphical icon may change during triggering. In addition, the manual NIBP selectable icon 596 may be disabled, such that a manual inflation of the cuff cannot be triggered by a user while the cuff is already undergoing an active inflation pattern as part of acquiring an updated blood pressure measurement.

Embodiments use a machine learning model or trained model as discussed herein to establish learned relationships between inputs to a medical monitoring system and outputs. Rather than using a conversion equation or calibration adjustments in combination with a conversion equation, the machine learning model learns many relationships that may not be readily apparent to a human observer. A trained machined learning model can improve result accuracy, particularly for conditions that are not well quantified through a conversion equation even when calibration is used. In order to get higher accuracy results, the machine learning model can be trained as further described herein. Further, in contrast to a conversion equation or a calibration function, the machine learning model may have unpredictable and dynamic outputs based on the training data and updates.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A noninvasive blood pressure monitoring system, comprising:
a noninvasive blood pressure sensor;
one or more patient sensors that generate sensor data; and
a monitoring device that receives the sensor data and comprising:
a memory storing a trained model; and
processing circuitry that provides the sensor data as input to the trained model and that generates a signal to activate the noninvasive blood pressure sensor based on an output of the trained model.

2. The system of claim 1, wherein the trained model is trained on patient data comprising monitored blood pressure and concurrently acquired patient sensor data.

3. The system of claim 2, wherein the signal is generated based on the model predicting that the sensor data corresponds to a change in blood pressure.

4. The system of claim 1, wherein the sensor data comprises a segment of a photoplethysmography signal.

5. The system of claim 1, wherein the sensor data comprises a physiological parameter.

6. The system of claim 1, wherein the sensor data comprises one or more of pulse oximetry monitoring data, carbon dioxide monitoring data, temperature data, or EEG data.

7. The system of claim 1, wherein the signal to activate the noninvasive blood pressure sensor causes a cuff of the noninvasive blood pressure sensor to inflate and to subsequently deflate as a part of blood pressure monitoring.

8. The system of claim 1, wherein the processing circuitry of the monitoring device operates to identify a type of the one or more patient sensors and to select the trained model from a plurality of trained models based on the identified type.

9. The system of claim 1, wherein the monitoring device operates to receive blood pressure data as a result of activating the noninvasive blood pressure sensor.

10. The system of claim 9, wherein the processing circuitry of the monitoring device operates to update the trained model using the blood pressure data and the sensor data.

11. A method of noninvasive blood pressure monitoring, the method comprising:
receiving sensor data of a patient from one or more patient sensors, the sensor data corresponding to a time window;
determining, using a trained model, that the sensor data is associated with a predicted change in blood pressure for the patient; and
triggering a noninvasive blood pressure sensor based on the predicted change in blood pressure.

12. The method of claim 11, comprising:
receiving new sensor data from the one or more patient sensors, the new sensor data corresponding to a subsequent time window after the time window;
determining, using the trained model, that the new sensor data is associated with a predicted stable blood pressure for the patient; and
not triggering the noninvasive blood pressure sensor based on the predicted stable blood pressure.

13. The method of claim 11, comprising:
receiving blood pressure data from the triggered noninvasive blood pressure sensor; and
determining that the predicted change in blood pressure is not present in the blood pressure data.

14. The method of claim 13, comprising: increasing a confidence threshold of the trained model associated with predicting subsequent changes in blood pressure based on the predicted change in blood pressure not being present in the blood pressure data.

15. The method of claim 13, comprising: switching activation of the noninvasive blood pressure sensor to timed intervals based on the predicted change in blood pressure not being present in the blood pressure data.
